(19) United States
(12) Patent Application Publication
(10) Pub. No.: US 2016/0159969 A1
(43) Pub. Date: Jun. 9, 2016

(54) EPOXY RESIN COMPOSITIONS

(71) Applicant: BLUE CUBE IP LLC, Midland, MI (US)

(72) Inventors: Chen CHEN, Shanghai (CN); Yan WU, Shanghai (CN); Yue SHEN, Shanghai (CN); Yurun YANG, Shanghaicn (CN)

(21) Appl. No.: 14/907,675

(22) PCT Filed: Jul. 26, 2013
(86) PCT No.: PCT/CN2013/080159

§ 371 Date: Jan. 26, 2016

(51) Int. Cl. C08G 59/24; C09D 163/00; C08G 59/50; C07D 303/26

(52) U.S. Cl. CPC C08G 59/245; C07D 303/26; C09D 163/00; C08G 59/504

(57) ABSTRACT

A novel modified epoxy compound that is capable of providing a coating composition with acceptable viscosity, short tack-free time and thy-hard time, and providing resultant coating films with good impact resistance; a process of preparing the modified epoxy compound; an epoxy resin composition comprising the modified epoxy compound; and a curable coating composition comprising the epoxy resin composition and a curing agent.

### FIELD OF THE INVENTION

The present invention relates to a modified epoxy compound and the process of its preparation. The present invention also relates to an epoxy resin composition comprising the modified epoxy compound and a curable coating composition comprising such epoxy resin composition.

### INTRODUCTION

Epoxy resins are widely used in coating applications such as anti-corrosion coatings. It is always desirable for coating compositions to have its solids content as high as possible in order to minimize the use of volatile organic compounds (VOCs). End users also expect to apply such coating compositions with incumbent application means such as spray guns, paint brushes or paint rollers.

Currently, widely used anti-corrosion coating compositions are based on solid epoxy resins and conventional curing agents such as polyamide, which can provide coating films with flexibility sufficient to meet industry requirements. However, such coating compositions usually only have up to about 50% volume solids, since large amounts of solvents are required to dissolve these solid epoxy resins. Compared to solid epoxy resins, conventional liquid epoxy resins have lower viscosity and are able to provide coating compositions with higher solids content, but the flexibility of the resultant coating films are usually poorer.

Attempts have been made to increase the solids content of coating compositions. For example, reactive and/or non-reactive diluents are added to coating compositions. Unfortunately, adding these diluents usually increases the cost of coating compositions and compromises their drying properties. The chemical resistance properties of the resultant coating films may also be compromised.

Therefore, it is desirable to provide an epoxy resin composition that is suitable for coating compositions with higher volume solids content than those compositions based on solid epoxy resins. It is also desirable that such coating compositions are able to provide coating films with better flexibility than conventional liquid epoxy resins without compromising the drying properties of the coating composition.

### SUMMARY OF THE INVENTION

The present invention provides a novel modified epoxy compound of Formula (I) that offers a solution to the problems described above. An epoxy resin composition comprising the modified epoxy compound has desirably low viscosity, and is capable of forming a coating composition with higher volume solids content than coating compositions based on solid epoxy resin. The coating composition of the present invention is able to provide the resultant coating films with improved front impact resistance and reverse impact resistance properties without compromising drying properties of the coating composition. In a first aspect, the present invention is a modified epoxy compound of Formula (I):

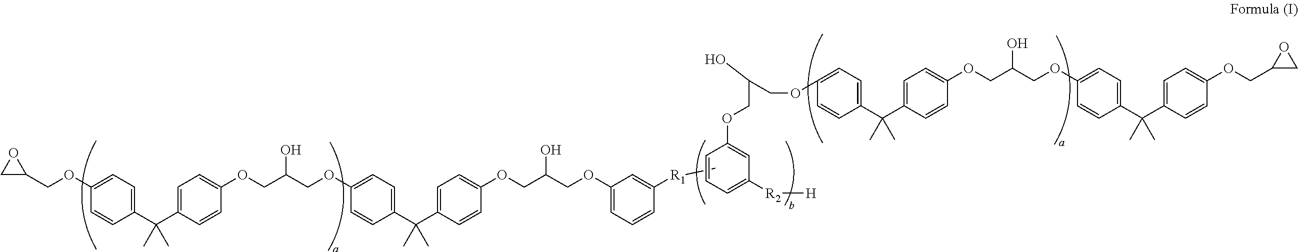

wherein each a is independently from 0 to 3; b is from 1 to 5; R₁ is a straight-chain or branched alkyl with 15 carbons containing 0 to 2 C═C bond(s); and R₂ is a straight-chain or branched alkyl with 15 carbons containing 0 to 3 C═C bond(s).

In a second aspect, the present invention is a process of preparing the modified epoxy compound of the first aspect. The process comprises:

(i) modifying cardanol by a Friedel-Crafts reaction in the presence of an acid catalyst, and (ii) reacting the resultant compound with a third epoxy compound of Formula (III):

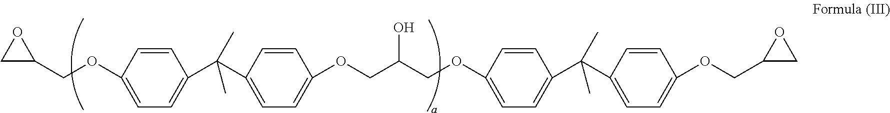

wherein a is independently from 0 to 3.

In a third aspect, the present invention is an epoxy resin composition comprising, based on the total weight of the epoxy resin composition,

(a) from 5 to 60 weight percent of the modified epoxy compound of the first aspect,

(b) from 5 to 60 weight percent of a second epoxy compound of Formula (II),

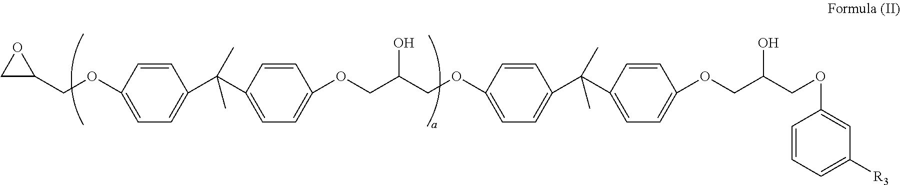

wherein R₃ is a straight-chain alkyl with 15 carbons containing 0 to 3 C═C bond(s) selected from the group consisting of —C₁₅H₃₁, —C₁₅H₂₉, —C₁₅H₂₇, and —C₁₅H₂₅; and a is independently from 0 to 3; and

(c) from 30 to 90 weight percent of a third epoxy compound of Formula (III):

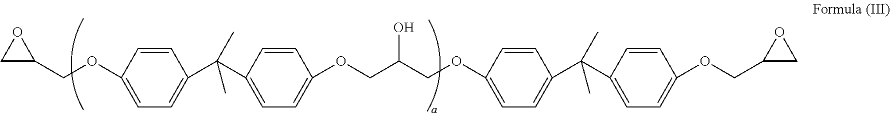

wherein a is independently from 0 to 3.

In a fourth aspect, the present invention is a curable coating composition comprising (I) the epoxy resin composition of the third aspect, and (II) a curing agent.

### DETAILED DESCRIPTION OF THE INVENTION

The modified epoxy compound of the present invention has the structure of the following Formula (I):

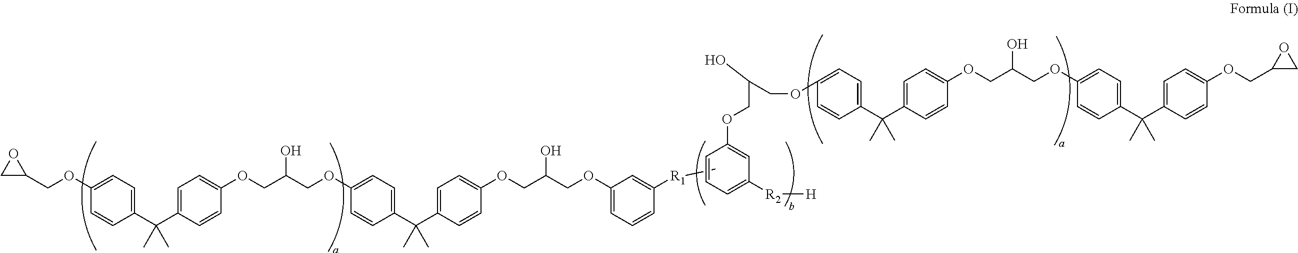

wherein each a is independently from 0 to 3; b is from 1 to 5; R₁ is a straight-chain or branched alkyl with 15 carbons containing 0 to 2 C═C bond(s); and R₂ is a straight-chain or branched alkyl with 15 carbons containing 0 to 3 C═C bond(s).

Preferably, a is from 0 to 1 and b is 1, 2 or 3. More preferably, a is 0 and b is 1.

R₁ may be a group selected from —C₁₅H₂₅—, —C₁₅H₂₇—, or —C₁₅H₂₉—.

R₂ may be a group selected from —C₁₅H₃₀, —C₁₅H₂₈—, —C₁₅H₂₆—, or —C₁₅H₂₄—.

The modified epoxy compound of the present invention may be a mixture of at least two different epoxy compounds all having the structure of Formula (I). For example, the modified epoxy compound may be (i) a mixture of a compound having Formula (I), wherein b is 1, and (ii) a compound having Formula (I), wherein b is 2, 3, 4 or 5, and preferably 2 or 3.

The modified epoxy compound of the present invention may have an epoxide equivalent weight (EEW) of 500 or more, 550 or more, or even 600 or more, and at the same time, 2,500 or less, 1,500 or less, 1,200 or less, or even 1,000 or less.

The epoxy resin composition of the present invention comprises the modified epoxy compound described above. The concentration of the modified epoxy compound in the epoxy resin composition may be, based on the total weight of the epoxy resin composition, 5 weight percent (wt %) or more, 8 wt % or more, or even 10 wt % or more, and at the same time, 60 wt % or less, 50 wt % or less, 40 wt % or less, or even 30 wt % or less.

The epoxy resin composition of the present invention may further comprise one or more second epoxy compounds of the following Formula (II):

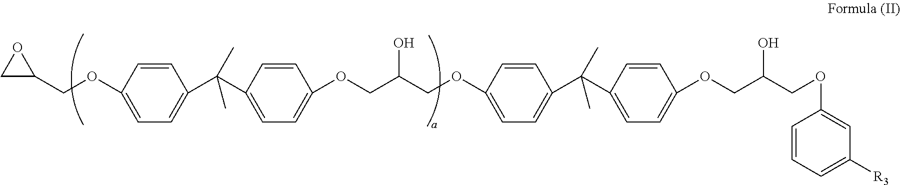

wherein R₃ is a straight-chain alkyl with 15 carbons containing 0 to 3 C═C bond(s) selected from the group consisting of —C₁₅H₃₁, —C₁₅H₂₉, —C₁₅H₂₇, and —C₁₅H₂₅; and a is independently from 0 to 3. Preferably, a is from 0 to 1, and more preferably 0.

The second epoxy compound of Formula (II) herein is also called “cardanol-modified epoxy compound.” The second epoxy compound may be a mixture of at least two different epoxy compounds of Formula (II).

The second epoxy compound useful in the present invention may have an EEW of 500 or more, 550 or more, or even 600 or more, and at the same time, 2,000 or less, 1,500 or less, or even 1,000 or less.

The concentration of the second epoxy compound in the epoxy resin composition may be, based on the total weight of the epoxy resin composition, 5 wt % or more, 8 wt % or more, or even 10 wt % or more, and at the same time, 60 wt % or less, 50 wt % or less, 40 wt % or less, or even 30 wt % or less.

The total combined concentration of the modified epoxy compound and the second epoxy compound in the epoxy resin composition may be, based on the total weight of the epoxy resin composition, 15 wt % or more, 20 wt % or more, or even 25 wt % or more, and at the same time, 60 wt % or less, 55 wt % or less, or even 50 wt % or less.

The epoxy resin composition of the present invention may further comprise one or more third epoxy compounds of the following Formula (III):

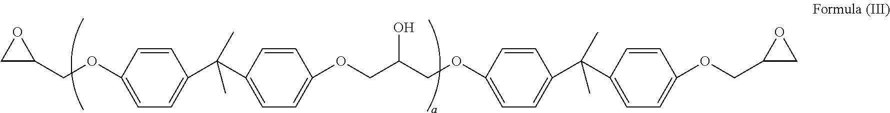

wherein a is independently from 0 to 3, and preferably from 0 to 1.

The third epoxy compound useful in the present invention may be a mixture of at least two different epoxy compounds all having the structure of Formula (III). The third epoxy compound useful in the present invention may have an EEW of 130 or more, 150 or more, 160 or more, 170 or more, 350 or more, 400 or more, or even 450 or more, and at the same time, 1,200 or less, 700 or less, 550 or less, 250 or less, 220 or less, 210 or less, or even 195 or less. The third epoxy compound may comprise a mixture of two or more epoxy compounds with different EEW.

Suitable commercially available third epoxy compounds useful in the present invention include, for example, D.E.R.™ 331 (D.E.R. is a trademark of The Dow Chemical Company), D.E.R. 332 , D.E.R. 330, D.E.R. 383, D.E.R. 671 epoxy resins all available from The Dow Chemical Company; and mixtures thereof.

The concentration of the third epoxy compound in the epoxy resin composition may be, based on the total weight of the epoxy resin composition, 30 wt % or more, 40 wt % or more, or even 50 wt % or more, and at the same time, 90 wt % or less, 85 wt % or less, or even 80 wt % or less.

The epoxy resin composition of the present invention may be a liquid mixture or a semi-solid mixture. The epoxy resin composition may have an EEW of 180 or more, 200 or more, or even 220 or more, and at the same time, 500 or less, 400 or less, 350 or less, or even 300 or less. In some embodiments, the epoxy resin composition has a viscosity of from 7,000 to 50,000 centipoises (cps), from 14,000 to 35,000 cps, or from 17,000 to 30,000 cps, according to the test method described in the Examples section below.

The process of preparing the modified epoxy compound of the present invention comprises: (i) modifying cardanol by a Friedel-Crafts reaction in the presence of an acid catalyst, and (ii) reacting the resultant compound with a third epoxy compound of Formula (III) described above (the third epoxy compound of Formula (III) is hereinafter referred to as “raw material epoxy resin”). In step (i), cashew nut shell liquid (“CNSL”) may be used, which mainly comprises cardanol and cardol. Cardanol is a mixture of phenols which contain one hydroxyl group and differ in the number of C═C bonds in the aliphatic side chain in the meta-position. The structure of cardanol is shown as follows:

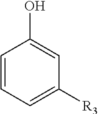

wherein R₃ is as previously defined with reference to Formula (II).

In the step (i) of preparing the modified epoxy compound of the present invention, the Friedel-Crafts reaction can be conducted according to known methods. The Friedel-Crafts reaction herein refers to the alkylation of the aromatic ring of one cardanol molecule with the double bond(s) on alkyl group (R₃ group) of another cardanol molecule. The Friedel-Crafts reaction is preferably conducted in the presence of an acid catalyst. Examples of suitable acid catalysts for the Friedel-Crafts reaction of cardanol include Lewis acids such as aluminium chloride, ferric chloride and boron trifluoride; Bronsted acids such as sulfuric acid, 4-toluene sulfonic acid, benzenesulfonic acid, methanesulfonic acid, and trifluoromethanesulfonic acid; or mixtures thereof. The acid catalyst useful in the Friedel-Crafts reaction may be used in an amount from 0.001 to 10 wt %, from 0.01 to 3 wt %, from 0.03 to 1.5 wt %, or from 0.05 to 1.5 wt %, based on the weight of cardanol. The Friedel-Crafts reaction may be conducted in the presence or absence of a solvent with the application of heating and mixing. The reaction temperature for the Friedel-Crafts reaction may be from 0 to 200° C., from 25 to 150° C., or from 50 to 100° C. Depending on the reaction temperature and desired conversion ratio of cardanol, the time for the Friedel-Crafts reaction may range from 5 minutes to 48 hours, from 20 minutes to 24 hours, or from 40 minutes to 12 hours. The obtained compound from the step (i) comprises a compound having the following structure of Formula (IV):

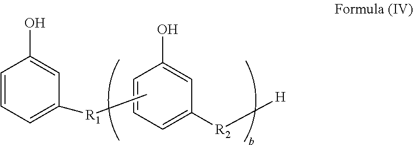

wherein R₁, R₂ and b are as previously defined with reference to Formula (I).

The compound of Formula (IV) herein is also called “cardanol oligomer”. The compound of Formula (IV) may be a mixture of a compound of Formula (IV) wherein b is 1 (“cardanol dimer”), a compound of Formula (IV) wherein b is 2 (“cardanol trimer”), and/or a compound of Formula (IV) wherein b is from 3 to 5. In some embodiments, the obtained compound from the step (i) comprises unreacted cardanol. The viscosity of the modified epoxy compound can be adjusted by controlling the conversion ratio of cardanol in the Friedel-Crafts reaction. Higher conversion ratio of cardanol tends to obtain more cardanol oligomers of Formula (IV) wherein b is 2 or higher.

In step (ii) of preparing the modified epoxy compound of the present invention, reacting the compound obtained from the step (i) with the raw material epoxy resin can be conducted according to known methods, for example, a modification reaction of an epoxy resin with phenols, wherein an active hydrogen atom is reacted with an epoxy group in the epoxy resin. In the step (ii), the active hydrogen atom(s) in hydroxyl group(s) of cardanol oligomer and, if present, unreacted cardanol, is reacted with epoxide group(s) of the raw material epoxy resin. The modification reaction described above may be conducted in the presence or absence of a solvent with the application of heating and mixing. The reaction temperature may be from 20 to 260° C., from 80 to 200° C., or from 100 to 180° C. In general, the time for completion of the modification reaction may range from 5 minutes to 24 hours, from 30 minutes to 8 hours, or from 30 minutes to 4 hours. A catalyst is preferably added in the modification reaction. Examples of suitable catalysts for the modification reaction include basic inorganic reagents, phosphines, quaternary ammonium compounds, phosphonium compounds, tertiary amines, and mixtures thereof. Preferred catalysts include sodium hydroxide (NaOH), potassium hydroxide (KOH), ethyl triphenyl phosphonium acetate, imidazole, or triethylamine. The catalyst useful in the modification reaction may be used in an amount from 0.01 to 3 wt %, from 0.03 to 1.5 wt %, or from 0.05 to 1.5 wt %, based on the total weight of the raw material epoxy resin. In some embodiments of the present invention, the compound obtained from the step (i), and the raw material epoxy resin are mixed in proper amounts as described above, and are dissolved and heated under conditions of the modification reaction as described above to form the modified epoxy compound of the present invention.

The epoxy resin composition of the present invention may be prepared substantially the same as the above described process for preparing the modified epoxy compound of Formula (I). In some embodiments, raw material cardanol in the step (i) of preparing the epoxy resin composition is preferably partially modified into the cardanol oligomer. In step (ii) of preparing the epoxy resin composition, both the cardanol oligomer and cardanol react with the raw material epoxy resin through the modification reaction described in the step (ii) of preparing the modified epoxy compound of Formula (I). The cardanol oligomer reacts with the raw material epoxy resin to form the compound of Formula (I). Cardanol reacts with the raw material epoxy resin to form the compound of Formula (II). The conversion ratio of cardanol in the step (i) may be from 5% to 70%, from 10% to 60%, or from 15% to 50%. The conversion ratio of cardanol is calculated by the peak area percentage change of the cardanol peak as determined by Gel Permeation Chromatography (GPC). The compound obtained from the step (i) to be reacted with the raw material epoxy resin may be used in an amount of 5 wt % or more, 10 wt % or more, or even 15 wt % or more, and at the same time, 60 wt % or less, 50 wt % or less, or even 40 wt % or less, based on the total weight of the compound obtained from the step (i) and the raw material epoxy resin.

The epoxy resin composition of the present invention can be cured by a curing agent (also known as “crosslinking agent” or “hardener”) having an active group being reactive with an epoxy group of the epoxy resin composition. Examples of suitable curing agents useful in the present invention include anhydrides, nitrogen-containing compounds such as amines and their derivatives, oxygen-containing compounds, sulfur-containing compounds, aminoplasts, polyisocyanates including blocked isocyanates, beta-hydroxyalkylamides, polyacids, organometallic acid-functional materials, and mixtures thereof. Amine-based curing agents such as polyamines are preferred. The curing agent may comprise one or more polyamine compounds. Examples of suitable polyamine compounds include an aliphatic polyamine, an alicyclic polyamine, an aromatic polyamine, a heterocyclic polyamine, polyamide, phenalkamine, amine adducts, or mixtures thereof. Preferably, the curing agent comprises a phenalkamine curing agent, for example, a phenalkamine compound, its adduct, or mixtures thereof. The phenalkamine compound may comprise a reaction product of cardanol, formaldehyde, and a polyamine such as ethylenediamine through the Mannich reaction. Suitable commercially available phenalkamine compounds useful in the present invention include for example CARDOLITE™ NC 541, CARDOLITE NC 541LV, and CARDOLITE NX 2015 hardeners available from Cardolite Cooperation; D.E.H™ 641 hardener available from The Dow Chemical Company; or mixtures thereof.

Curing the epoxy resin composition of the present invention may be carried out, for example, at a temperature in a range of from −10 to 300° C., from −5 to 250° C., from 10 to 220° C., or from 21 to 25° C. Generally, the time for curing or partially curing the epoxy resin composition is from 2 seconds to 24 days, from 0.5 hour to 7 days, or from one hour to 24 hours. It is also operable to partially cure the epoxy resin composition of the present invention and then complete the curing process at a later time.

The epoxy resin composition of the present invention may be used in various applications such as coatings, adhesives, electrical laminates, structural laminates, structural composites, filament windings, moldings, castings, encapsulations, pultrusion and any application where impact resistance is desirable.

The curable coating composition of the present invention comprises (I) the epoxy resin composition described above, and (II) the curing agent described above. The curing agent may be used in a sufficient amount to cure the curable coating composition. For example, the molar ratio of a) total epoxy functionality of the epoxy resin composition to b) total active hydrogen functionality of the curing agent in the curable coating composition may be 10:1 or lower, 5:1 or lower, 3:1 or lower, or even 2:1 or lower, and at the same time, 1:2 or higher, 1:1.5 or higher, 1:0.9 or higher, or even 1:0.5 or higher.

The curing agent in the curable coating composition of the present invention may comprise a curing catalyst to increase the curing speed between the epoxy resin composition and the curing agent. Examples of suitable curing catalysts include salicylic acid, tris(dimethylaminomethyl)phenol, or mixtures thereof. The curing catalyst may be present in an amount from 0 to 10 wt %, from 0.1 to 8 wt %, or from 0.5 to 5 wt %, based on the total weight of the curing agent.

The curing agent in the curable coating composition of the present invention may also comprise an accelerator. Examples of suitable accelerators include benzyl alcohol, nonyl phenol, or mixtures thereof. The accelerator may be present in an amount of 0 to 55 wt %, 0.5 to 50 wt %, or 5 to 30 wt %, based on the total weight of the curing agent.

The curable coating composition of the present invention may also include organic solvents. The components mentioned above present in the curable coating composition may be dissolved or dispersed in an organic solvent. Examples of suitable solvents include alcohols such as n-butanol; ketones; glycols such as ethylene glycol, propylene glycol, and butyl glycol; glycol ethers such as propylene glycol monomethyl ether and ethylene glycol dimethyl ether; xylene; toluene; acetates such as glycol ether acetates; mineral oil; naphthas; and mixtures thereof. The organic solvent is generally present in an amount of from 5 to 60 wt %, or from 8 to 30 wt %, based on the total weight of the curable coating composition.

The curable coating composition of the present invention may include inorganic extender and/or pigments. Examples of suitable inorganic extender and/or pigments include iron oxides, calcium carbonate, precipitated silica, magnesium carbonate, talc, anticorrosive pigments such as zinc phosphate and zinc powder, titanium dioxide, iron oxides, carbon black, metallic materials including metalloid materials, feldspar powder, or mixtures thereof. The concentration of the inorganic extenders and/or pigments in the curable coating composition is generally from 0 to 65 wt %, from 5 to 60 wt %, or from 10 to 40 wt %, based on the total weight of the curable coating composition.

The curable coating composition of the present invention may also comprise one or more additional film-forming resins that are different from the epoxy compounds in the epoxy resin composition of the present invention. Examples of the additional film-forming resins include polyurethane, acrylics, alkyds, polyester, polyether, polysiloxane, and mixtures thereof.

In addition to the foregoing components, the curable coating composition of the present invention may further comprise one or more following additives: anti-foaming agents, plasticizers, leveling agents, wetting agents, dispersants, thixotropic agents, adhesion promoters, rheology modifiers, anti-oxidants, diluents and grind vehicles. When present in the curable coating composition, these additives may be in an amount of 0.001 to 10 wt %, or from 0.01 to 2 wt %, based on the total weight of the curable coating composition.

The curable coating composition may be prepared with techniques known in the coating art. The curable coating composition can be prepared by admixing the epoxy resin composition and the curing agent, which are preferably dissolved in the organic solvent. Other optional components including for example inorganic extenders and/or pigments and/or other optional additives may also be added, as described above. Components in the curable coating composition may be mixed in any order to provide the curable coating composition of the present invention. Any of the above-mentioned optional components may also be added to the composition during or prior to the mixing to form the curable coating composition.

The curable coating composition of the present invention can be similarly cured under the conditions used for curing the epoxy resin composition described above to form a coating film. In some embodiments, the curable coating composition is cured by a phenalkamine curing agent at room temperature.

The curable coating composition of the present invention can be applied by incumbent means including brushing, dipping, rolling and spraying. The curable coating composition is preferably applied by spraying. The standard spray techniques and equipment for spraying such as air-atomized spraying, air spraying, airless spraying, high volume low pressure spraying, and electrostatic spraying such as electrostatic bell application, and either manual or automatic methods can be used.

In some embodiments, the curable coating composition of the present invention has a volume solids content of 65% or more, or even 70% or more. “Volume solids content” is determined by the test method described in the Examples section below.

The curable coating composition of the present invention has several advantages. Coating films made from the curable coating composition of the present invention have better front and reverse impact resistance as compared to incumbent coating compositions that do not comprise the modified epoxy compound of the present invention upon curing by the same curing agents. The curable coating composition of the present invention has shorter tack-free time and dry-hard time compared to coating compositions that comprise the cardanol-modified epoxy compound and the same curing agents in the absence of the modified epoxy compound of the present invention.

The curable coating composition of the present invention can be applied to, and adhered to, various substrates. Examples of suitable substrates include wood, metals, plastics, foams, stones, including elastomeric substrates, glass, fabrics, concrete, cementious substrates, or substrates that are found on motor vehicles. The curable coating composition is suitable for various coating applications, such as low VOC coatings, marine and protective coatings, automotive coatings, wood coatings, coil coatings, plastic coatings, powder coatings, can coatings, and civil engineering coatings. The curable coating composition is particularly suitable for heavy duty anticorrosive primers such as zinc-rich coatings. The curable coating composition can be used alone, or in combination with other coatings to form multi-layer coatings. For example, a multi-layer coating may comprise the curable coating composition of the present invention as a primer, a tie coat and, optionally, a topcoat.

### EXAMPLES

The following examples illustrate embodiments of the present invention. All parts and percentages in the examples are by weight unless otherwise indicated. The following materials are used in the examples:

Cardanol is available from Shanghai Meidong Biomaterials Company.

p-toluenesulfonic acid, available from Sinopharm Chemical Reagent Co., Ltd., is used as a catalyst.

D.E.R. 331 resin, available from The Dow Chemical Company, is a diglycidyl ether of bisphenol A that has an EEW of 182-192.

D.E.R. 671-X75 epoxy resin solution, available from The Dow Chemical Company, comprises 75 wt % of a diglycidyl ether of bisphenol A having an EEW of 420-550 and 25 wt % of xylene, based on the total weight of the epoxy resin solution.

D.E.R. 671 resin, available from The Dow Chemical Company, is a diglycidyl ether of bisphenol A having an EEW of 420-550.

Ethyl triphenyl phosphonium acetate is used as a catalyst and is available from The Dow Chemical Company.

CARDOLITE NC 541LV hardener, available from Cardolite Corporation, is a phenalkamine hardener having an amine value of 310-345 mg KOH/g. The amine value is the number of milligrams of potassium hydroxide (KOH) required for reacting with perchloric acid (HClO₄) which is used to neutralize 1 gram of amine resin.

Xylene is a solvent available from Sinopharm.

Feldspar powder is available from Pingxing Xintai Feldspar Powder Plant.

CRAYVALLAC™ Super rheology modifier is an amide wax rheology modifier available from Cary Valley Ltd.

BYK™-A 530 is a defoamer available from BYK.

VERSAMID™ 115 hardener, available from BASF, is a polyamide hardener having an amine value of 230-246 mg KOH/g.

The following standard analytical equipment and methods are used in the Examples.

### Epoxide Equivalent Weight (EEW) Analysis

A standard titration method is used to determine percent epoxide in various epoxy resins. The titration method used is similar to the method described in Jay, R. R., “Direct Titration of Epoxy Compounds and Aziridines”, Analytical Chemistry, 36, 3, 667-668 (March 1964). In the present adaptation of this method, the carefully weighed sample (sample weight ranges from 0.17-0.25 gram) was dissolved in dichloromethane (15 mililiter (mL)) followed by the addition of tetraethylammonium bromide solution in acetic acid (15 mL). The resultant solution treated with 3 drops of crystal violet indicator (0.1% wt/vol in acetic acid) was titrated with 0.1 N perchloric acid in acetic acid on a Metrohm 665 Dosimat titrator (Brinkmann). Titration of a blank consisting of dichloromethane (15 mL) and tetraethylammonium bromide solution in acetic acid (15 mL) provided correction for solvent background. Percent epoxide and EEW are calculated using the following equations:

% Epoxide=[(mL titrated sample)−(mL titrated blank)]×(0.4303)/(gram sample titrated)EEW=43023/[% Epoxide]

### Volume Solids Content

The volume solids content of a coating composition is calculated as follows. The total volume of pigment and inorganic extender in the coating composition is denoted as Vₚ. The total volume of non-volatile solids except pigment and inorganic extender in the coating composition (also known as “volume of solid binder”) is denoted as V_{b}. The total volume of the coating composition (also known as “total wet paint volume”) is denoted as V_{w}. The volume solids content of the coating composition is measured according to the following equation:

Volume solids=[(Vₚ+V_{b})/V_{w}]×100%

### Viscosity

The viscosity of an epoxy resin composition or a paint formulation is measured at 25° C. using a Brookfield CAP 2000+ viscometer, 6# rotator, and 400 revolutions per minute (rpm).

### Drying Properties

A BYK drying recorder is used to record tack-free time and dry-hard time of a coating composition according to the ASTM D 5895 method. The coating composition to be evaluated is coated on a glass panel and then put on to the BYK drying timer for drying at room temperature.

### Impact Resistance

The front and reverse impact resistance properties of coating films are measured according to the ASTM D 2794 method. A coating composition to be evaluated is directly sprayed onto a Q panel and dried at room temperature for 7 days to form a coating film. The obtained coating film has an average thickness of 60-80 microns. A 2 pound standard weight is used. By gradually increasing the height distance the weight drops, the distance at which failure occurs is recorded.

### Preparation of COMPOUND I

Two hundred grams of cardanol and 1 gram of p-toluenesulfonic acid were added into a three-neck flask under nitrogen atmosphere. The resultant mixture were heated to 100° C. and kept at this temperature for about 8 hours until the conversion ratio of cardanol was about 28% as monitored by GPC by calculating the peak area percentage change of the cardanol peak. The obtained COMPOUND I was analyzed by ¹³CNMR and GPC. The ¹³CNMR results showed new peaks at δ 144.5 and δ 36.1 in ¹³CNMR, which gave the evidence for the generation of the new carbon (on the aromatic ring of cardanol)-carbon (on the alkyl group of cardanol) bond in cardanol oligomers. The GPC results also demonstrated the generation of higher molecular weight products. The obtained COMPOUND I had a polystyrene (PS) equivalent weight average molecular weight (Mw) of 582 and a polydispersity index (PDI) of 1.24 according to GPC. Liquid chromatography/mass spectrometry (LS/MS) detected that cardanol dimer (m/z 595) existed in the obtained COMPOUND I.

### Preparation of COMPOUND II

Two hundred grams of cardanol and 1 gram of p-toluenesulfonic acid were added into a three-neck flask under nitrogen atmosphere. The resultant mixture were heated to 120° C. and kept at this temperature for 4 hours to obtain COMPOUND II. In the above reaction, the conversion ratio of cardanol was about 50% as calculated from the peak area percentage change of the cardanol peak as measured by GPC. The obtained COMPOUND II was analyzed by ¹³CNMR and GPC. The ¹³CNMR results showed new peaks at δ 144.5 and δ 36.1 in ¹³CNMR, which gave the evidence for the generation of the new carbon (on the aromatic ring of cardanol)-carbon(on the alkyl group of cardanol) bond in cardanol oligomers. The GPC results also demonstrated the higher Mw products generation. The obtained COMPOUND II had a PS equivalent Mw of 1126 and a PDI of 1.71 according to GPC calibrated. LS/MS detected that cardanol dimer (m/z 595) existed in the obtained COMPOUND II.

### Examples (Exs) 1-6

Epoxy resin compositions of Exs 1-6 were prepared based on formulations shown in Table 1. COMPOUND I or COMPOUND II was prepared according to the procedure described above, then each mixed with D.E.R. 331 resin under nitrogen atmosphere in a three-neck flask. After the mixture reached 90° C., 5,000 ppm ethyl triphenyl phosphonium acetate (in 70 wt % methanol solution) was added as a catalyst. The resultant mixture was heated to 180° C. and kept at this temperature for 2 hours to obtain the epoxy resin compositions. The epoxy resin compositions obtained from the above procedure were analyzed by GPC, and details of these epoxy resin compositions were reported in Table 2.

### Comparative Example (Comp Ex) A

90 wt % D.E.R. 331 resin and 10 wt % cardanol were mixed under nitrogen atmosphere in a flask, where weight percentage is based on the total weight of raw materials. After the mixture reached 90° C., 200 ppm ethyl triphenyl phosphonium acetate (in 70 wt % methanol solution) was added as a catalyst. The resultant mixture was heated to 180° C. and kept at this temperature for 2 hours to obtain the epoxy resin composition of Comp Ex A. Details of the epoxy resin composition obtained from the above procedure were reported in Table 2.

TABLE 1

Raw Material (Weight percentage based on the total
weight of raw materials)

| | D.E.R. 331 resin | COMPOUND I | COMPOUND II |
|---|---|---|---|
| Ex 1 | 90 | 10 | |
| Ex 2 | 85 | 15 | |
| Ex 3 | 80 | 20 | |
| Ex 4 | 90 | | 10 |
| Ex 5 | 85 | | 15 |
| Ex 6 | 80 | | 20 |

TABLE 2

| | Epoxy Resin Composition (Weight percentage relative to the total | | | | | | | |
|---|---|---|---|---|---|---|---|---|
| | weight of epoxy resin composition) | | | | | | | |
| | | | | | | | Comp | D.E.R. |
| | Ex 1 | Ex 2 | Ex 3 | Ex 4 | Ex 5 | Ex 6 | Ex A** | 671 resin |
| Compound of | 9 | 20 | 26 | 11 | 16 | 30 | 0 | — |
| Formula (I) | | | | | | | | |
| Compound of | 16 | 20 | 25 | 11 | 16 | 18 | 22 | — |
| Formula (II) | | | | | | | | |
| Compound of | 75 | 60 | 49 | 78 | 68 | 52 | 78 | 100 |
| Formula (III) | | | | | | | | |
| EEW* | 226 | 261 | 294 | 226 | 261 | 294 | 226 | 470 |
| Viscosity (cps) | 12,405 | 18,375 | 22,950 | 16,875 | 22,050 | 36,600 | 11,340 | Solid |

*EEW of the epoxy resin composition
**Concentration of each component is calculated according to EEW change.

As shown in Table 2, viscosities of the epoxy resin compositions of Exs 1-6 were all much lower than that of D.E.R. 671 resin which is solid at room temperature. When D.E.R. 331 resin was modified with the same dosage of cardanol oligomer (Exs 1 and 4) or cardanol (Comp Ex A), viscosities of the resultant epoxy compositions of Exs 1 and 4 were similar or slightly higher than that of Comp Ex A, but still acceptable.

### Exs 7-12 Coating Compositions

Coating compositions of Exs 7-12 were prepared by mixing CARDOLITE NC 541LV hardener, xylene with the epoxy resin compositions of Exs 1-6 obtained above, respectively, based on formulations shown in Table 3.

### Comp Ex B Coating Composition

The coating composition of Comp Ex B was prepared by mixing CARDOLITE NC 541LV hardener, xylene with the epoxy resin composition of Comp Ex A obtained above, based on formulations shown in Table 3.

### Comp Ex C Coating Composition

The coating composition of Comp Ex C was prepared by mixing CARDOLITE NC 541LV hardener, xylene with D.E.R. 331 epoxy resin, based on formulations shown in Table 3.

TABLE 3

| | | Coating Composition (Weight percentage relative to the total | | | | | | | |
|---|---|---|---|---|---|---|---|---|---|
| | | weight of the coating composition) | | | | | | | |
| | | | | | | | | Comp | Comp |
| | | Ex 7 | Ex 8 | Ex 9 | Ex 10 | Ex 11 | Ex 12 | Ex B | Ex C |
| Epoxy Resin | Ex 1 | 58.0 | | | | | | | |
| Composition | Ex 2 | | 60.8 | | | | | | |
| | Ex 3 | | | 63.2 | | | | | |
| | Ex 4 | | | | 58.0 | | | | |
| | Ex 5 | | | | | 60.8 | | | |
| | Ex 6 | | | | | | 63.2 | | |
| | Comp Ex A | | | | | | | 58.0 | |
| | D.E.R. 331 resin | | | | | | | | 53.9 |

| | | | | | | | | |
|---|---|---|---|---|---|---|---|---|
| CARDOLITE NC 541LV | 32.0 | 29.2 | 26.8 | 32.0 | 29.2 | 26.8 | 32.0 | 36.1 |
| Xylene | 10.0 | 10.0 | 10.0 | 10.0 | 10.0 | 10.0 | 10.0 | 10.0 |

Drying properties of the above coating compositions and impact resistance properties of coating films formed from these coating compositions were evaluated according to the test methods described above and were reported in Table 4.

As shown in Table 4, the coating composition of Comp Ex B showed a tack-free time of 7.5 hours, and a dry-hard time of 12 hours. In contrast, the coating compositions of Exs 7-12 showed a tack-free time of 7 hours or less, and a dry-hard time of 10 hours or less, which are much shorter than those of the coating composition of Comp Ex B.

As shown in Table 4, the coating compositions of Exs 7-12 provided coating films with much better front and reverse impact resistance compared to the coating compositions of Comp Exs B and C.

TABLE 4

| | Drying Properties (hour) | | | Impact Resistance | |
|---|---|---|---|---|---|
| | Set to touch | Tack-free | Dry-hard | (centimeter (cm)) | |
| | Time | Time | Time | Front | Reverse |
| Ex 7 | 5.5 | 7 | 9.5 | 30 | 5 |
| Ex 8 | 5 | 6.7 | 8.75 | 35 | 30 |
| Ex 9 | 5.4 | 6.9 | 10 | 50 | 40 |
| Ex 10 | 4.5 | 5.2 | 6.3 | 35 | 5 |
| Ex 11 | 4.5 | 5.7 | 7.5 | 55 | 35 |
| Ex 12 | 4.5 | 5.5 | 7.4 | 70 | 60 |
| Comp Ex B | 6.2 | 7.5 | 12 | 25 | 3 |
| Comp Ex C | — | — | — | 10 | <3 |

### Ex 13 and Comp Ex D Paint Formulations

Paint Formulations of Ex 13 and Comp Ex D were prepared based on formulations shown in Table 5. Part A was prepared by dispersing inorganic extender and pigment and other additives into an epoxy resin composition and solvent by a high-speed dispersing machine at room temperature. Part B was prepared by mixing VERSAMID 115 curing agent and xylene at room temperature while stirring until the resultant mixture became homogenous. Part A and Part B obtained above were mixed to form the paint formulations of Ex 13 and Comp Ex D, respectively. Viscosities of the paint formulations were measured according to the test method described above.

As shown in Table 5, the volume solids content of the paint formulation of Ex 13 is 65%, compared to a volume solids content of 50% for the paint formulation of Comp Ex D. The above two paint formulations showed a similar initial viscosity of 670 cps and 620 cps for the paint formulations of Comp Ex D and Ex 13, respectively. As shown in Table 2, epoxy resin compositions of Exs 1-5 all showed even lower viscosity than that of Ex 6. The epoxy resin compositions of Exs 1-6 can be formulated to paint formulations with higher volume solids content and lower VOC content compared to D.E.R. 671 resin.

TABLE 5

| | | Paint Formulations | |
|---|---|---|---|
| | | (wt % based on the total | |
| | | weight of paint formulation) | |
| | | Comp Ex D | Ex 13 |
| Part A | Feldspar powder | 29.4 | 38.6 |
| | D.E.R. 671-X75 | 26.5 | 0 |
| | Epoxy Resin Composition | 0 | 21.0 |
| | of Ex 6 | | |
| | Butanol | 8.7 | 6.2 |
| | CRAYVALLAC Super | 0.8 | 0.9 |
| | rheology modifier | | |
| | Xylene | 23.9 | 13.9 |
| | BYK-A 530 | 0.2 | 0.2 |
| Part B | VERSAMID 115 | 7.8 | 14.5 |
| | hardener | | |
| | Xylene | 2.7 | 4.7 |

1. A modified epoxy compound of Formula (I): 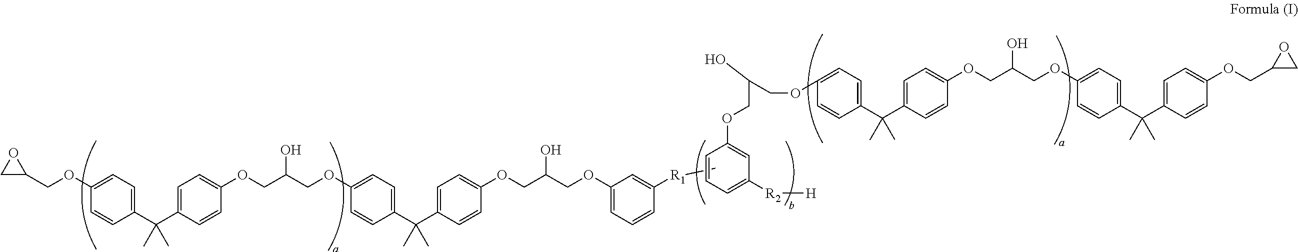
wherein each a is independently from 0 to 3; b is from 1 to 5; R₁ is a straight-chain or branched alkyl with 15 carbons containing 0 to 2 C═C bond(s); and R₂ is a straight-chain or branched alkyl with 15 carbons containing 0 to 3 C═C bond(s).

2. The modified epoxy compound of claim 1, wherein b is from 1 to 3.

3. The modified epoxy compound of claim 1, wherein R₁ is —C₁₅H₂₅—, —C₁₅H₂₇—, or —C₁₅H₂₉— group.

4. A process of preparing the modified epoxy compound of claim 1, comprising:
(i) modifying cardanol by a Friedel-Crafts reaction in the presence of an acid catalyst, and (ii) reacting the resultant compound with a third epoxy compound of Formula (III): 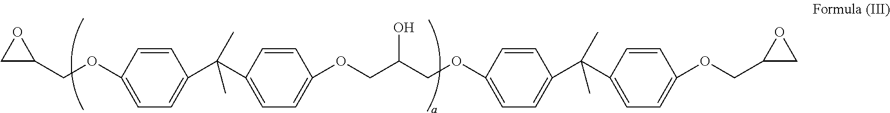
wherein a is independently from 0 to 3.

5. An epoxy resin composition comprising, based on the total weight of the epoxy resin composition,
(a) from 5 to 60 weight percent of the modified epoxy compound of any one of claims 1-3,
(b) from 5 to 60 weight percent of a second epoxy compound of Formula (II), 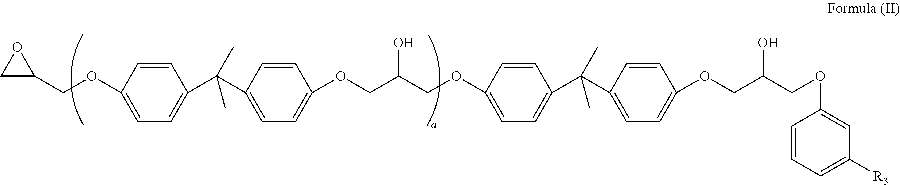
wherein R₃ is a straight-chain alkyl with 15 carbons containing 0 to 3 C═C bond(s) selected from the group consisting of —C₁₅H₃₁, —C₁₅H₂₉, —C₁₅H₂₇, and —C₁₅H₂₅;
and a is independently from 0 to 3; and
(c) from 30 to 90 weight percent of a third epoxy compound of Formula (III): 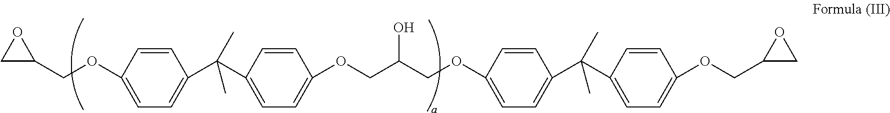
wherein a is independently from 0 to 3.

6. The epoxy resin composition of claim 5, wherein the epoxy resin composition comprises: (a) from 5 to 40 weight percent of the modified epoxy compound, (b) from 10 to 30 weight percent of the second epoxy compound, and (c) from 40 to 85 weight percent of the third epoxy compound.

7. The epoxy resin composition of claim 5, wherein the total combined concentration of the modified epoxy compound and the second epoxy compound is from 15 to 60 weight percent, based on the total weight of the epoxy resin composition.

8. The epoxy resin composition of claim 5, wherein the total combined concentration of the modified epoxy compound and the second epoxy compound is from 25 to 55 weight percent, based on the total weight of the epoxy resin composition.

9. The epoxy resin composition of claim 5, wherein the third epoxy compound has an epoxide equivalent weight of from 150 to 210.

10. The epoxy resin composition of claim 5, wherein the epoxy resin composition has an epoxide equivalent weight of from 200 to 300.

11. A curable coating composition comprising (I) the epoxy resin composition of claim 5, and (II) a curing agent.

12. The curable coating composition of claim 11, wherein the curing agent comprises a phenalkamine compound.

13. The curable coating composition of claim 11, further comprising solvent, an accelerator, a curing catalyst, an inorganic extender and/or pigment, a filler, or mixtures thereof.

14. The curable coating composition of claim 11, wherein the molar ratio of a) total epoxy functionality of the epoxy resin composition to b) total active hydrogen functionality of the curing agent is from 10:1 to 1:2.

15. The modified epoxy compound of claim 1, wherein b is from 1 to 3; and R₁ is —C₁₅H₂₅—, —C₁₅H₂₇—, or —C₁₅H₂₉— group.

16. The epoxy resin composition of claim 6, wherein the total combined concentration of the modified epoxy compound and the second epoxy compound is from 15 to 60 weight percent, based on the total weight of the epoxy resin composition.

17. The curable coating composition of claim 12, wherein further comprising solvent, an accelerator, a curing catalyst, an inorganic extender and/or pigment, a filler, or mixtures thereof

18. The epoxy resin composition of claim 6, wherein the total combined concentration of the modified epoxy compound and the second epoxy compound is from 25 to 55 weight percent, based on the total weight of the epoxy resin composition.

19. The epoxy resin composition of claim 6, wherein the third epoxy compound has an epoxide equivalent weight of from 150 to 210.

20. The epoxy resin composition of claim 6, wherein the epoxy resin composition has an epoxide equivalent weight of from 200 to 300.
